(19) United States
(12) Patent Application Publication
(10) Pub. No.: US 2024/0226493 A9
(43) Pub. Date: Jul. 11, 2024

(54) RESPIRATORY OR SURGICAL HUMIDIFIER AND COMPONENTS THEREOF

(71) Applicant: Fisher & Paykel Healthcare Limited, Auckland (NZ)

(72) Inventors: Wenjie Robin Liang, Auckland (NZ); Yintao Yu, Auckland (NZ)

(21) Appl. No.: 18/547,318

(22) PCT Filed: Feb. 23, 2022
(86) PCT No.: PCT/IB2022/051563

§ 371 Date: Aug. 21, 2023

Related U.S. Application Data
(60) Provisional application No. 63/154,197, filed on Feb. 26, 2021.

(51) Int. Cl. A61M 16/16; A61M 16/00; A61M 39/08; A61M 39/12

(52) U.S. Cl. CPC A61M 16/161; A61M 16/0003; A61M 16/024; A61M 39/08; A61M 39/12; A61M 2016/0027; A61M 2016/0039; A61M 2205/3334; A61M 2205/3372; A61M 2205/3561; A61M 2205/584

(57) ABSTRACT

A respiratory or surgical humidifier comprising a heater base and an electrical connector. The heater base is configured to receive a humidifier chamber and supply humidified gases to a breathing tube when coupled with the chamber. The heater base also comprises a control circuit. The electrical connector is provided for electrically coupling the control circuit to a breathing tube. The electrical connector comprises a first power line, a second power line, and a data line configured to allow serial communication between the control circuit and one or more digital devices. Also provided is a humidifier system, a breathing tube, a humidifier chamber and an electrical connector for a humidifier.

### FIELD OF THE DISCLOSURE

The present disclosure relates to respiratory and/or surgical humidifiers, and respiratory or breathing assistance systems for gases to be supplied to a patient or user via a gas supply tube.

### BACKGROUND

Respiratory apparatuses are used in various environments, such as hospital, medical facilities, residential care, palliative care or home environments. For a range of respiratory applications, it is beneficial to humidify gases being supplied to a patient or user. These applications include where the gases are for breathing by the patient or user and/or where the gas is being supplied during surgery to the patient or user. In the case of breathing gases in a non-invasive mode when the inspired gas passes through the upper airway, such as when gas is delivered to the patient or user via a face or nasal mask, the humidity increases patient or user comfort, improves the patient's or user's tolerance to the non-invasive ventilation (NIV), and the humidified gases are less prone to drying out the tissues (for example, the nasal mucosa) of the airway of the patient or user. In the case of surgical gases when the gases are delivered to a surgical site of the patient or an invasive mode when the gases delivered to the patient bypass the upper airway, humidification of the gases has been found to improve patient comfort and provide physiological benefits, such as improved mucus transport, can be necessary for patient or user safety, such as for preventing airway obstruction due to inspissation of airway secretion, disruption of the airway epithelium (or mesothelium in surgical applications), and/or for improving post-operative outcomes. In the case of high flow therapy, humidified gases are delivered to the patient or user at high flows through an unsealed interface. The patient or user may be spontaneously breathing or may be apneic, such as under anesthesia. A flow therapy apparatus with a humidifier can be used to deliver high flow gases and the therapy apparatus may control characteristics such as for example gases flow, including flow rate, temperature, pressure, humidity, supplementary gases concentration, and the like. In the case of positive airway pressure therapy (PAP) therapy, a PAP therapy apparatus that includes a blower and a humidifier can be used to provide pressure therapy, for example, continuous positive airway pressure therapy (CPAP), to the user.

### SUMMARY

Humidification systems include breathing circuit components like breathing tubes, patient interfaces and various couplings and other tube sections that connect to the breathing tube. These can include electronic components that require power to function. Often, a breathing tube will include a heating element such as a coiled wire that receives power from the heater base. Sensors are also sometimes provided in components of the breathing circuit, for example to sense temperature or airflow. Sensors can also be used to sense the ambient temperature and control power to the heating element of the breathing tube based on the ambient temperature. Multiple sensors may provide redundancy in the case of sensor failure.

The present disclosure includes examples of humidifiers and components that can employ a connector providing an electrical connection between a control circuit in a heater base of the humidifier and a breathing tube having a first power line, a second power line, and a data line. The power lines provide power to components of, or connected to, the breathing tube. The data line can allow serial communications to be performed between the control circuit and one or more digital devices, which can be in the connector itself, in the breathing tube, in a component coupled to the breathing tube, or in any combination of these. The digital devices can be parasitically powered over the data line. Any number of digital devices can be connected to the data line. Data can be gathered from digital devices at various locations in the humidifier system without the need to provide additional power and data wires in the connector or in one or more separate leads. This can enable backwards compatibility with other connector cables and breathing tubes, which in turn minimizes production, design and compliance testing costs and time. Avoiding the need for an additional lead also maintains the ease of connecting components of the humidifier system and reduces the chance of user error caused by user plugging a lead in incorrectly or failing to plug it in. Avoiding the need for an extra lead also reduces the possibility of the lead tangling in use and reduces the time taken to set up the humidifier. The data line can be used to communicate with a digital temperature sensor which can be provided along with an analog temperature sensor. This can improve reliability of, and add redundancy to, temperature measurements. Digital communications with devices in the humidifier system can also be addressed to any one or other number of devices connected to the data line. Communications with digital devices in the humidifier system can also be checked for integrity.

In some configurations a respiratory or surgical humidifier can comprise: a heater base configured to receive a humidifier chamber and supply humidified gases to a breathing tube when coupled with the chamber, the heater base comprising a control circuit; and an electrical connector for electrically coupling the control circuit to a breathing tube, the electrical connector comprising: a first power line; a second power line; and a data line configured to allow serial communication between the control circuit and one or more digital devices.

In some configurations the one or more digital devices comprise one or more integrated circuits.

In some configurations the one or more digital devices comprise one or more sensors.

In some configurations the one or more sensors can comprise one or more digital temperature sensors.

In some configurations the respiratory or surgical humidifier can further comprise one or more analog temperature sensors.

In some configurations one or more of the digital temperature sensors and/or one or more of the analog temperature sensors can be configured to sense an ambient temperature.

In some configurations one or more of the digital devices can be located in the connector.

In some configurations the connector can comprise a first end, a second end and a housing intermediate the first end and the second end wherein one or more of the digital devices is located in the housing.

In some configurations one or more of the analog temperature sensors can be located in the housing.

In some configurations one or more of the digital temperature sensors and one or more of the analog temperature sensors can be provided on a common printed circuit board (PCB) in the housing.

In some configurations the control circuit can be configured to determine ambient temperature using an output from the digital temperature sensor and an output from the analog temperature sensor.

In some configurations the control circuit can be configured to control an amount of power delivered to the breathing tube over the power lines based on the determined ambient temperature.

In some configurations the control circuit can be configured to detect a fault when an output of the digital temperature sensor or a value derived from the output of the temperature sensor differs from an output of the analog temperature sensor or a value derived from the output of the analog temperature sensor by an amount greater than a first difference threshold for a period greater than a first time threshold.

In some configurations the control circuit can be configured to detect a fault when an output of the digital temperature sensor or a value derived from the output of the temperature sensor differs from an output of the analog temperature sensor or a value derived from the output of the analog temperature sensor by an amount greater than a second difference threshold for a period greater than a second time threshold.

In some configurations the one or more digital devices can comprise one or more sensors selected from the group consisting of: a humidity sensor; a pressure sensor; an airflow sensor; a voltage sensor; a current sensor; a battery monitor; and a chemical sensor.

In some configurations the one or more digital devices can comprise one or more processors or data storage devices.

In some configurations one or more of the digital devices can be configured to be parasitically powered.

In some configurations one or more of the digital devices that can be parasitically powered can be configured to receive electrical energy over the data line.

In some configurations the control circuit and digital devices can be configured to communicate on the data line wherein the data line acts a bus.

In some configurations the control circuit and digital devices can be configured to communicate using a 1-wire communication protocol.

In some configurations one or more of the digital devices can be located in the chamber, the breathing tube, and/or in an intermediate tube section between the chamber and the tube or between the tube and a patient interface.

In some configurations the connector can comprise a Zener diode connected between the data line and the second power line for overvoltage protection.

In some configurations the connector can be coupled to the control circuit by a wired connection.

In some configurations the connector can be configured to be coupled to the control circuit by a plug.

In some configurations the connector can be configured to wirelessly couple to the control circuit.

In some configurations the connector can further comprise a variable visual indicator for indicating status information to a user.

In some configurations the variable visual indicator can comprise a light-emitting diode (LED).

In some configurations the output of the visual indicator can be controlled based on an operating, configurational or environmental condition of the humidifier.

In some configurations controlling the output of the visual indicator can comprise controlling the output color or temporal illumination pattern of the LED.

In some configurations the output of the visual indicator can be controlled based on a fault condition of the humidifier.

In some configurations the visual indicator can be configured to produce a plurality different outputs corresponding to a plurality of respective fault conditions.

In some configurations the respiratory or surgical humidifier can further comprise a component configured to be electrically connected to the connector, the component comprising: a first power line configured to be electrically connected to the first power line of the connector; a second power line configured to be electrically connected to the second power line of the connector; and a data line configured to be electrically connected to the data line of the connector; wherein the second power line of the component can be configured to provide a reference for communications on the data line of the component.

In some configurations the component can be a breathing tube, an intermediate component of the breathing circuit, a humidifier chamber, or an intermediate connector.

In some configurations the connector can have only three electrical terminals at an end that couples to the breathing tube, each terminal provided on a respective one of the first power line, second power line, and data line.

In some configurations a respiratory or surgical humidifier can comprise: a heater base comprising a control circuit; a chamber configured to be received by the heater base; a breathing tube configured to pneumatically couple with the chamber; and an electrical connector for electrically coupling the control circuit to the breathing tube, the electrical connector comprising: a first power line; a second power line; and a data line configured to allow serial communication between the control circuit and one or more digital devices.

In some configurations the one or more digital devices can comprise one or more integrated circuits.

In some configurations the one or more digital devices can comprise one or more sensors.

In some configurations the one or more digital devices can comprise one or more temperature sensors.

In some configurations the one or more digital devices can comprise one or more sensors selected from the group consisting of: a humidity sensor; a pressure sensor; an airflow sensor; a voltage sensor; a current sensor; a battery monitor; and a chemical sensor.

In some configurations the one or more digital devices can comprise one or more processors or data storage devices.

In some configurations one or more of the digital devices can be configured to be parasitically powered over the data line.

In some configurations the respiratory or surgical humidifier can further comprise an intermediate tube section that can be configured to be pneumatically coupled to the breathing tube between the breathing tube and the chamber.

In some configurations the respiratory or surgical humidifier can further comprise an intermediate tube section that can be configured to be pneumatically coupled to the breathing tube between the breathing tube and a patient interface.

In some configurations the connector can be configured to be mechanically connected to an electrical interface of the breathing tube.

In some configurations the connector can be configured to be mechanically connected to an electrical interface of the chamber.

In some configurations the connector can be configured to be mechanically connected to an electrical interface of the intermediate tube section that can be configured to be pneumatically coupled to the breathing tube between the breathing tube and the chamber.

In some configurations the connector can be configured to be mechanically connected to an electrical interface of the intermediate tube section that can be configured to be pneumatically coupled to the breathing tube between the breathing tube and the patient interface.

In some configurations the connector can be releasably connectable to the electrical interface of the breathing tube independently of pneumatic connections of the breathing tube.

In some configurations the breathing tube can be configured to simultaneously pneumatically couple to the chamber and electrically couple to the chamber, a patient interface, or an intermediate component connected between the breathing tube and the chamber or between the breathing tube and a patient interface.

In some configurations the one or more digital devices can comprise one or more temperature sensors in the connector.

In some configurations the one or more digital devices can comprise one or more temperature sensors in the breathing tube.

In some configurations the one or more digital devices can comprise one or more temperature sensors in the chamber.

In some configurations the one or more digital devices can comprise one or more temperature sensors in an intermediate component connected between the breathing tube and the chamber or between the breathing tube and a patient interface.

In some configurations the breathing tube can comprise a heating element and the control circuit can be configured to control an amount of power provided to the heating element based on the output(s) of one or more of the temperature sensors.

In some configurations the one or more digital devices can comprise three digital devices and the control circuit can be configured to selectively communicate with two or more of the three digital devices.

In some configurations there can be no sensor provided at the end of the breathing tube configured to connect to a patient interface or in an intermediate component configured to be connected between the breathing tube and a patient interface.

In some configurations there can be no sensor provided at the end of the breathing tube configured to connect to the chamber or in an intermediate component configured to be connected between the breathing tube and the chamber.

In some configurations the one or more digital devices can comprise one or more digital devices configured to communicate wirelessly with the control circuit or with each other.

In some configurations the respiratory or surgical humidifier can further comprise a component configured to be electrically connected to the connector, the component comprising: a first power line configured to be electrically connected to the first power line of the connector; a second power line configured to be electrically connected to the second power line of the connector; a data line configured to be electrically connected to the data line of the connector; wherein the second power line of the component is configured to provide a reference for communications on the data line of the component.

In some configurations the component can be the breathing tube, an intermediate component of the breathing circuit, a humidifier chamber, or an intermediate connector.

In some configurations the connector can have only three terminals at an end that couples to the breathing tube, each terminal provided on a respective one of the first power line, second power line, and data line.

In some configurations a breathing tube for a respiratory or surgical humidifier can comprise: a breathing conduit including a pneumatic coupling at one end to couple the breathing conduit with a humidifier chamber of a respiratory or surgical humidifier; a heater element configured to heat gases within the breathing conduit; and an electrical interface for electrically coupling the breathing tube to a heater base of a respiratory or surgical humidifier, the electrical interface comprising: a first power line electrically connected to the heater element; a second power line electrically connected to the heater element; and a data line configured to allow serial communication between a control circuit in the heater base and one or more digital devices.

In some configurations one or more of the digital devices can be located in the breathing tube.

In some configurations the one or more of the devices that can be located in the breathing tube can comprise an integrated circuit.

In some configurations the one or more of the devices that can be located in the breathing tube can comprise a temperature sensor.

In some configurations one or more of the digital devices can be located in a component that the breathing tube is configured to be coupled to.

In some configurations the second power line can be configured to provide a reference for communications on the data line.

In some configurations the electrical interface can have only three terminals, each of the terminals provided on a respective one of the first power line, the second power line, and the data line.

In some configurations a respiratory or surgical humidifier can comprise: a heater base configured to receive a humidifier chamber and supply humidified gases to a breathing tube when coupled with the chamber, the heater base comprising a control circuit; and an electrical connector for electrically coupling the control circuit to a breathing tube, the electrical connector comprising a data line configured to allow serial communication between the control circuit and one or more processing or data storage devices.

In some configurations one or more of the processing or data storage devices can be configured to be parasitically powered over the data line.

In some configurations the connector can have only three terminals at an end that can be configured to couple to the breathing tube, one of the terminals provided on the data line, another of the terminals provided on a first power line and the other of the terminals provided on a second power line.

In some configurations a respiratory or surgical humidifier system can comprise: a heater base comprising a control circuit; a chamber configured to be received by the heater base; a breathing tube configured to pneumatically couple with the chamber; and an electrical connector for electrically coupling the control circuit and the breathing tube, the electrical connector comprising a data line configured to allow serial communication between the heater base and one or more processing or data storage devices.

In some configurations one or more of the processing or data storage devices can be configured to be parasitically powered over the data line.

In some configurations the connector can have only three terminals at an end that couples to the breathing tube, one of the terminals connected to the data line, another of the terminals connected to a first power line and the other of the terminals connected to a second power line.

In some configurations a breathing tube for a respiratory or surgical humidifier can comprise: a breathing conduit including a pneumatic coupling at one end to couple the breathing conduit with a humidifier chamber of a respiratory or surgical humidifier; a heater element configured to heat gases within the breathing conduit; one or more processing or data storage devices; and an electrical interface for electrically coupling the breathing tube to a heater base of the respiratory or surgical humidifier, the electrical interface comprising a data line configured to allow serial communication between the one or more processing or data storage devices and a control circuit in the heater base.

In some configurations one or more of the processing or data storage devices can be configured to be parasitically powered over the data line.

In some configurations the electrical interface can have only three terminals, one of the terminals provided on the data line, another of the terminals provided on a first power line and the other of the terminals provided on a second power line.

In some configurations a humidifier chamber configured to be received by a heater base of a respiratory or surgical humidifier can comprise: a gases inlet for receiving gases to be humidified; a gases outlet for supplying humidified gases to a breathing tube of the humidifier; and one or more electrical lines for electrically coupling the heater base to the breathing tube, each electrical line having a terminal proximate the gases outlet; wherein the one or more electrical lines is integral with the chamber.

In some configurations the one or more electrical lines can comprise a data line configured to allow serial communications between a control circuit of the heater base and one or more digital devices.

In some configurations the one or more electrical lines can further comprise a first power line and a second power line.

In some configurations an electrical connector for a respiratory or surgical humidifier can comprise: a first electrical interface comprising two power terminals and a data terminal; a second electrical interface comprising two power terminals and a data terminal, the power and data terminals of the second electrical interface being electrically connected to respective power and data terminals of the first electrical interface via two power lines and a data line; and a digital device electrically connected to the data line and one of the power lines, the digital device configured to perform serial communications over the data line.

Where the construction “and/or” is used it refers to the inclusive form of “or” known as the Boolean OR operator, meaning “(and) or (or)”. Unless the context implies otherwise, where the term “or” is used it refers to the inclusive form of “or”.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1A illustrates an example respiratory or surgical humidifier system.

FIG. 1B illustrates an example humidifier heater base of the respiratory or surgical humidifier system of FIG. 1A.

FIG. 1C illustrates an example respiratory or surgical humidifier heater base and connector of the respiratory or surgical humidifier system of FIG. 1A.

FIG. 2 schematically illustrates an example respiratory humidifier system in use.

FIG. 3 illustrates an example respiratory humidifier system.

FIG. 4 illustrates an end of an example connector for a respiratory or surgical humidifier system.

FIG. 5 illustrates an example connector for a respiratory or surgical humidifier system.

FIG. 6 illustrates example components of the connector of FIG. 5.

FIG. 7 illustrates an example electrical circuit for the connector of FIG. 5.

FIG. 8 schematically illustrates a simplified example electrical circuit of a respiratory or surgical humidifier.

### DETAILED DESCRIPTION

Although certain embodiments and examples are described below, those of skill in the art will appreciate that the disclosure extends beyond the specifically disclosed embodiments and/or uses and to obvious modifications and equivalents thereof. Thus, it is intended that the scope of the disclosure herein disclosed should not be limited by any particular embodiments described below. For example, component values and operating parameters are examples only and are not limiting.

### Example Respiratory or Surgical Humidifier

The present disclosure provides examples of a respiratory humidifier configured to supply humidified and/or heated gas to a patient or user in multiple modes. The modes for the respiratory humidifier can include at least an invasive mode (for example, for patients with a bypassed airway) and a non-invasive mode (for example, for patients or users with breathing masks). Each mode can have a number of humidity settings, which can be expressed as a dew point or absolute humidity. The respiratory humidifier is controlled to deliver, at an outlet of the humidification chamber and/or the patient end of the gas supply tube, humidified gases having a dew point (or absolute humidity) at or near a predetermined humidity level. For example, a user can select a setting appropriate for the current mode of operation. A number of humidity settings may be provided, for example, the humidity settings may be equivalent to a dew point of 37 degrees Celsius, 31 degrees Celsius, 29 degrees Celsius, 27 degrees Celsius, or others. The humidity setting equivalent to a dew point of 37 degrees Celsius may be suitable for invasive therapy (i.e., where the patient's upper airways are bypassed) whereas the other humidity settings may be suitable for non-invasive therapy, although the humidity settings may not be restricted to a particular type of therapy. Alternatively, each humidity setting may be continuously variable between upper and lower limits. A lower humidity setting may be selected by the user to reduce condensation or “rain-out” in the gas supply tube or to improve patient comfort, or a higher humidity setting may be selected to improve physiological benefits. Some respiratory humidifier systems disclosed herein can also include a high flow mode, unsealed mode, or any other modes known to those of skill in the art. The disclosed devices and components may be similarly applied in a surgical humidifier which may be used, for example, in laparoscopic or open surgery.

High flow therapy as discussed herein is intended to be given its typical ordinary meaning, as understood by a person of skill in the art, which generally refers to a respiratory assistance system delivering a targeted flow of humidified respiratory gases via an intentionally unsealed patient interface with flow rates generally intended to meet or exceed inspiratory flow of a patient. Typical patient interfaces include, but are not limited to, a nasal or tracheal patient interface. Typical flow rates for adults often range from, but are not limited to, about fifteen liters per minute to about sixty liters per minute or greater. Typical flow rates for pediatric patients (such as neonates, infants and children) often range from, but are not limited to, about one liter per minute per kilogram of patient weight to about three liters per minute per kilogram of patient weight or greater. High flow therapy can also optionally include gas mixture compositions including supplemental oxygen and/or administration of therapeutic medicaments. High flow therapy is often referred to as nasal high flow (NHF), humidified high flow nasal cannula (HHFNC), high flow nasal oxygen (HFNO), high flow therapy (HFT), or tracheal high flow (TIM), among other common names. For example, in some configurations, for an adult patient ‘high flow therapy’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with an adult patient, a neonatal, infant, or child patient, may deliver gases to the patient at a flow rate of between about 1 LPM and about 100 LPM, or at a flow rate in any of the sub-ranges outlined above.

High flow therapy can be effective in meeting or exceeding the patient's inspiratory demand, increasing oxygenation of the patient and/or reducing the work of breathing. Additionally, high flow therapy may generate a flushing effect in the nasopharynx such that the anatomical dead space of the upper airways is flushed by the high incoming gases flow. The flushing effect can create a reservoir of fresh gas available of each and every breath, while minimizing re-breathing of carbon dioxide, nitrogen, etc.

The patient interface for use in a high flow therapy can be a non-sealing interface to prevent barotrauma, which can include tissue damage to the lungs or other organs of the patient's respiratory system due to difference in pressure relative to the atmosphere. The patient interface can be a nasal cannula with a manifold and nasal prongs, and/or a face mask, and/or a nasal pillows mask, and/or a nasal mask, and/or a tracheostomy interface, or any other suitable type of patient interface.

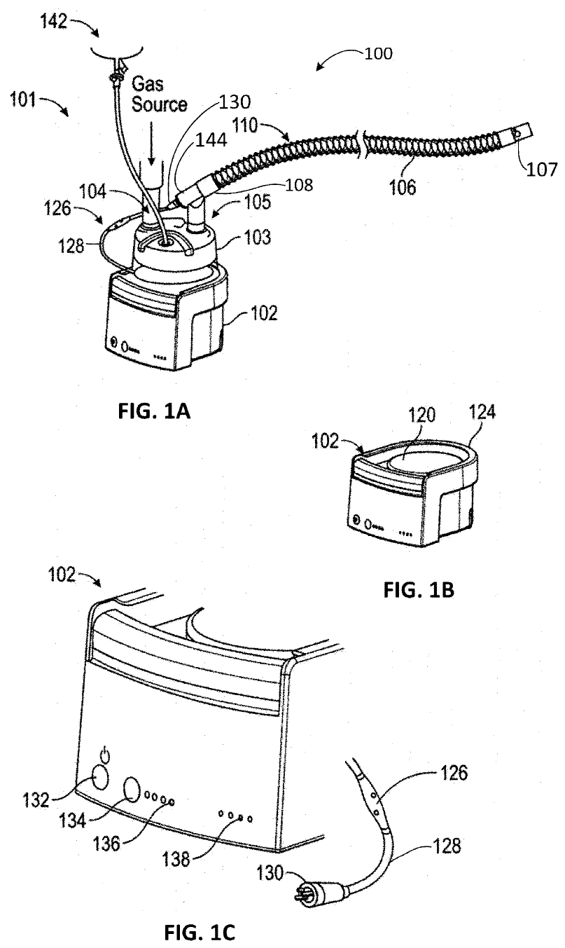

Referring to FIGS. 1A and 1C, an example respiratory humidifying system 100 can include a heater base 102 having a heat transfer body, which in this example is in the form of heater plate 120 (see FIG. 1B) having a substantially planar top surface. The heat transfer body may take different shapes including shapes that at least partially encompass the humidifier chamber 103. The heater base 102 and chamber 103 (along with the electrical connector 128 in this example) make up a humidifier 101 that acts to humidify gases for supply to a patient via the breathing circuit of the system 100. The heater plate 120 can have an electric heating element therein or in thermal contact therewith. Optionally one or more electrical insulation layers can be located between in the heater plate and the heater element. The heater element can be a base element (or a former) with a wire wound around the base element. The wire can be a nichrome wire (also known as nickel-chrome or chrome-nickel, being any of various alloys of nickel and chromium and sometimes other elements). The heater base 102 can have a housing and a control circuit (for example, a microprocessor-based controller) contained within the housing for controlling the supply of power to the heating element(s) of the heater plate 120.

The humidifier heater plate 120 can have at least one heater plate temperature sensor. An analog or digital temperature sensor may be used. The temperature sensor/s may be a temperature transducer, thermocouple, infrared sensor, a temperature sensor that produces a temperature signal based on the resistance profile of the heating element, a negative temperature coefficient thermistor, a positive temperature coefficient thermistor or other suitable type of sensor. The temperature sensor/s can measure a temperature of the heater plate 120. The temperature sensor can be in electrical communication with the control circuit in the heater base 102 so that the control circuit can monitor the temperature of the heater plate 120.

The humidifier chamber 103 can be removably received and retained on the heater base 102, such that the humidifier chamber base is positioned in contact with the heater plate 120 in the heater base 102. Referring to FIG. 1B, which illustrates an example of the heater base 102 of FIG. 1A, the heater base 102 can have a collar 124 for engaging with a flange on the humidifier chamber 103, such as shown in FIG. 1A. The collar 124 defines a lip that engages a flange of the humidifier chamber 103 to retain the humidifier chamber 103 in an operative position on the heater base 102. The humidifier chamber 103 can include a thermally conductive base. When engaged with the heater base 102, the conductive base of the humidifier chamber 103 can be in contact with a part of the heater plate 120, such as an upper surface of the heater plate 120. Water inside the chamber 103 is heated when a power signal is sent to the heating element to energize the heating element. The chamber 103 can also be connected to a water source 142, which can add water to the chamber 103 when the water is low or completely out in the chamber 103. Adding of water can be manually performed, such as upon a warning from the system 100 that there may be a low water or water-out condition, or automatically performed, such as using a float valve connected to a water supply.

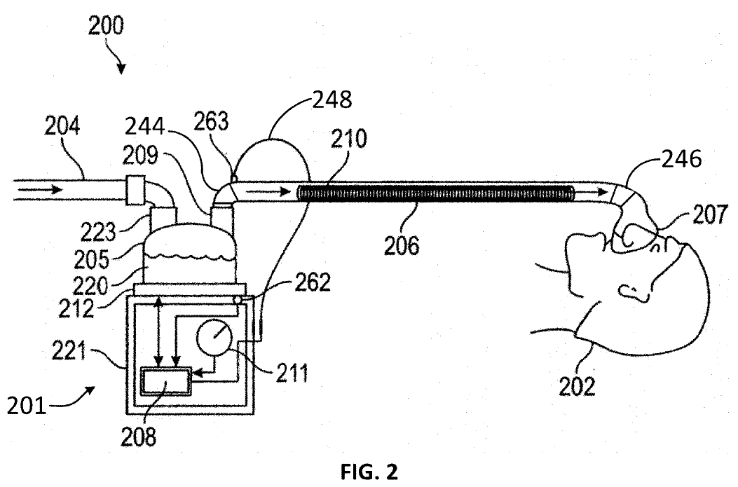

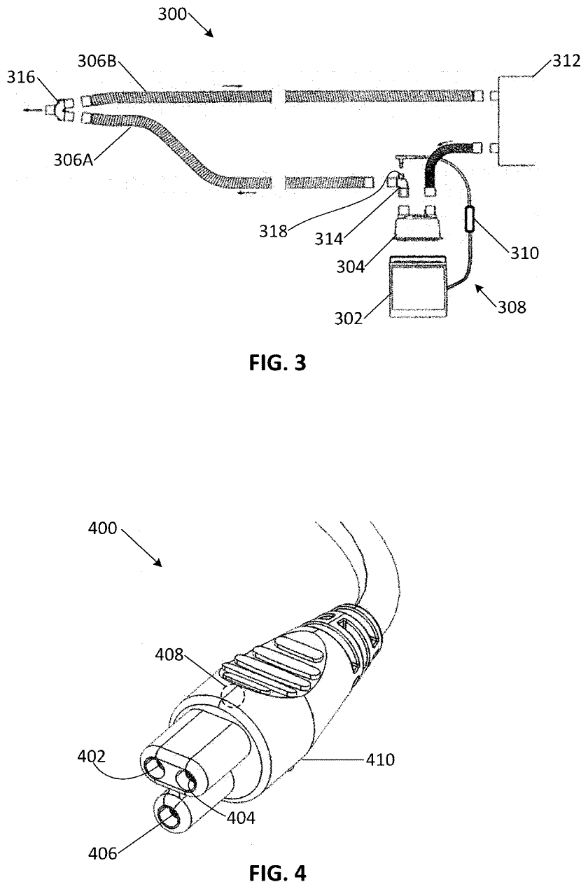

The gases to be humidified can include one or more of air, oxygen, anesthetic, other auxiliary gases, carbon dioxide, or any mixture of gases. The gases can be supplied to the humidifier chamber 103 through a gases inlet 104, which can be connected to a gas source, such as a ventilator, in the case of CPAP therapy a CPAP blower, or a remote source. For high flow therapy, a blower or further alternatively a wall source with a flow and/or pressure regulator can supply the gases. The humidifier chamber 103 also includes a gases outlet 105, which can connect to a breathing circuit which includes the breathing tube 106 of FIG. 1A. The breathing circuit can convey humidified and heated gases along a conduit to a patient or user. A patient end 107 of the breathing tube 106 can connect to a patient interface, such as a nasal cannula or a nasal mask. The breathing tube 106 can also connect to other types of patient or user interfaces, such as a full-face mask, total-face mask, nasal pillows mask, endotracheal tube, or others, or a surgical cannula or diffuser for surgical applications. The base end 108 of the breathing tube 106 can connect to the chamber 103 to receive humidified gases from the chamber 103. The breathing tube 106 can connect to the patient interface or the chamber 103 directly or via one or more intermediate components such as intermediate tube sections (illustrated in FIGS. 2 and 3). The breathing tube 106 includes a tube heating element 110. The breathing tube and other components to which it is coupled can have data and/or power lines in them to allow data communications to digital devices, and/or powering of components, in the breathing tube or in components coupled to the breathing tube 106.

A tube heating element 110 (such as one or more heater wires embedded in the breathing tube wall, contained within the breathing tube, or wrapped around the outside of the breathing tube) can be provided in the breathing tube 106. The tube heating element 110 reduces condensation and ensures the temperature and/or humidity of gases is maintained in a predetermined range, for example keeping the temperature of gases in the tube above a selected dew point. The tube heating element 110 is in electrical communication with the control circuit in the heater base 102 via connector 128. The connector 128 can provide power to the breathing tube 106 as well as allowing for data communication between the control circuit in the heater base 102 and one or more digital devices of the system 100. The connector 128 in this example is in the form of a cable, but it could take other forms such as a ribbon or a length of rigid electrical conduit. The connector could also be integral with a component such as the chamber.

The connector 128 can mechanically connect directly to the breathing tube 106 or to another component such as the chamber 103 or a component intermediate the breathing tube and the patient interface or chamber 103. In the case that the connector 128 does not connect directly to the breathing tube, electrical connectivity between the connector 128 and the breathing tube can be provided via the component to which it does mechanically connect. In the example of FIG. 1A, the connector 128 is mechanically and electrically connected to electrical interface 144 of the breathing tube 106. A similar electrical interface could be provided on another component (e.g. the chamber or intermediate component) in the case that the connector 128 couples to a component other than the breathing tube 106. As shown in FIG. 1C, the connector 128 can include a releasable end connector—in this case a female plug 130 which could couple to a corresponding male socket—for electrically connecting to the breathing tube 106. The particular plug shown is just one example of a suitable releasable end connector and various types of plug or socket or other releasable connector could be used. This allows for simple assembly and disassembly of the system. In particular, the connector 128 can be easily disconnected from the breathing tube 106 (or other component) to remove or replace parts of the system 100 such as the breathing tube or chamber and connected to the breathing tube 106 (or other component) to install parts. The coupling of the connector 128 to the breathing tube or any other part of the breathing circuit can be independent of the pneumatic couplings of the breathing circuit. In one example, the connector could be integral with a chamber of the humidifier system. In one example, the connector could include wires embedded in a side wall of the chamber. In other examples, the connector could be glued or welded or otherwise attached to the chamber. The connector could include terminals on or near the outlet of the chamber that the breathing tube would be coupled to. The terminals could electrically connect to corresponding terminals of the electrical interface on the breathing tube or on an intermediate component between the chamber and breathing tube. The electrical interface could be integrated with the pneumatic coupling of the breathing tube or intermediate component to provide a combined electrical and pneumatic coupling. The connector could also include terminals on the base or side of the chamber. These could electrically connect to corresponding terminals on the heater base.

The connector 128 can connect to the control circuit in various ways. The connector 128 can be permanently wired to the control circuit, for example by soldering. Alternatively, the connector 128 can be non-permanently connected to the control circuit by a releasable connection to the heater base 102, allowing the connector 128 to be removed from the heater base 102. For example, a releasable end connector like a plug could be provided at the heater base end of the connector 128 to couple to a corresponding socket in the heater base 102. In another example, wireless power transfer could be provided between the connector 128 and the base 102. This could allow wireless transmission of power and data signals between the heater base 102 and the connector 128. The wireless coupling could employ inductive or capacitive coupling to transfer power via magnetic and/or electric fields. Data signals could be modulated onto the power signal or received via another wireless communication channel such as a Bluetooth or NFC communication channel.

In one example, an intermediate connector could be provided. The intermediate connector could be configured to connect between one connector and another connector, between one connector and the heater base, between one connector and the breathing tube, between one connector and an intermediate component of the breathing circuit, or between one connector and the chamber. The intermediate connector could include two electrical interfaces, each having two power terminals and a data terminal. Two power lines could connect the power terminals of one interface to the power terminals of the other interface. A data line could connect the data line of one interface to the data line of the other interface. A digital device (such as any of the digital devices discussed herein) could be connected to the data line and configured to perform serial communications over the data line. The digital device could also be parasitically powered over the data line or in another way as discussed herein. The intermediate connector could be coupled into the electrical path between the heater base and another part of an existing humidifier system to add a digital device to the system. Two or more such intermediate connectors could also be added to provide additional digital devices to the system. The intermediate connectors could include different types of digital devices to provide different capabilities to the humidifier system. For example, one intermediate connector could have a digital temperature sensor, another could have a pressure sensor, and another could have a processor. In this way many additional devices may be easily integrated simply by connecting new intermediate connectors.

Digital devices can be located in various parts of the humidifier system 100. For example, the housing 126 of the connector 128 can house one or more digital devices.

Digital devices can also be located in the breathing tube 106 or in a component coupled to the breathing tube 106. The digital devices can be provided as integrated circuits. In some examples, the digital devices are sensors such as temperature sensors, temperature loggers, humidity sensors, pressure sensors, airflow sensors, voltage sensors, current sensors, chemical sensors or battery monitors. Temperature sensors may be used to sense the ambient temperature or the temperature of the breathable gases for supply to the patient, for example in order to control power provided to the heating element 110 of the breathing tube 106. A pressure sensor may be used to sense inhalation and exhalation of the patient. For example, the pressure sensor may be in a pneumatic line that is coupled between the gases source and a patient end of the breathing tube 106 or a component coupled to the patient end of the breathing tube 106.

The tube heating element 110 is controlled by the controller in the heater base 102, including the controlling of power to the tube heating element 110. The connector 128 can include at least one ambient temperature sensor in housing 126 (which may be an infrared detector, a negative temperature coefficient thermistor or a positive temperature coefficient thermistor), which can allow the system 100 to adjust the tube heating element 110 power and/or heater plate power to compensate for ambient temperatures or changes in the ambient temperature. The ambient temperature sensor/s can alternatively be located anywhere that is exposed to the ambient air.

As shown in FIG. 1C, a front panel of the heater base 102 can include a plurality of user controls and indicators, such as a power button 132, a humidity setting push button 134, and a plurality of (such as three, four, five or more) humidity settings indicators 136 (which can include LED lights) next to the humidity setting button 134. The locations, shapes, and sizes of the user controls and indicators are not limiting. The humidity level can be adjusted by pressing the humidity setting button 134, which can also be a momentary push button. The humidity setting button 134 could be any mechanism, for example, a push button, switch, dial, or a touch-sensitive interface. The front panel can also include a plurality of alarm indicators 138 (which can include LED lights) to indicate the following non-limiting examples of conditions: “water out” condition (including low water and water-out), connector 128 not connected, audible alarm muted, and a “See Manual” indication used to indicate that a fault has occurred within the system 101.

The system 100 can be suitable for providing respiratory therapy for different purposes, such as for critical care (for example, in the hospital) and home care. The system 100 is suitable for providing invasive, non-invasive and high flow therapies for both adult and pediatric patients.

An exemplary use of the humidifier system is discussed with reference to the simplified schematic humidifier system 200 of FIG. 2. In this use, the humidifier 201 receives gases from gas supply conduit 204 via inlet 223 of chamber 205. The gases are humidified with water evaporated from liquid water 220 and exit the chamber 205 via outlet 209. The humified gases are provided to a patient 202 via a breathing circuit which includes breathing tube 206, patient interface 207, and intermediate tube sections 244 and 246. While in the tube 206 the humidified gases receive heat energy from the heating element 210 in the breathing tube 206. As will be discussed in more detail with reference to FIGS. 3, 6 and 7, one or more temperature sensors can be provided to sense ambient temperature and/or breathing gases temperature. These sensed temperatures can be provided to the control circuit and used to control power to the heating element 210 of the breathing tube 206 and/or the power to a heater plate 212 of the heater base 221. A sensor 262 is also shown in the heater base 221 in this example. This can be used to sense the temperature of the heater plate 212 and provide an output representative of the sensed temperature to the control circuit 208 for closed-loop control and/or to sense a low water or water-out condition.

The heater base 221 includes heater plate 212, and user controls 211 and control circuit 208, which can be a microprocessor-based controller. Connector 248 is also provided to electrically couple the control circuit 208 to the breathing tube 206. In the configuration shown, the connector 248 is connected to the breathing tube at electrical interface 263. In other configurations, the connector 248 could be connected to the chamber 205, the intermediate component 244 between the breathing tube 206 and the chamber 205, or the intermediate component 246 between the breathing tube 206 and the patient interface 207. Intermediate components such as 244 and 246 may be disposable or reusable and sterilizable. The intermediate components 244 and 246 can be connected to the breathing tube 206, chamber 205, and/or patient interface 207 permanently or removably and by various means including for example a press/friction fit, glue, overmoulding, snap fit, screw coupling, and/or bayonet coupling. A power supply can power the heating element 210 over power lines provided in the connector 248. The control circuit can control the power provided from the power supply to the heating element 210. As will be discussed in more detail with reference to FIGS. 4 to 8, the connector 248 also allows for serial communications between the control circuit 208 and one or more digital devices. In some examples, the digital devices could be provided with wireless communications transmitters, receivers and/or transceivers to communicate wirelessly with the control circuit or with each other. These could form part of a WiFi or internet of things (“IOT”) network.

FIG. 3 shows an alternative humidifier system to that of FIG. 1A. In this humidifier system 300, the breathing circuit includes both an inspiratory breathing tube 306A and an expiratory breathing tube 306B. Gases for inspiration are provided from the gas source 312 to the chamber 304 where they are humidified and pass on to the inspiratory breathing tube 306A. The gas source 312 can be any suitable gas source including for example a ventilator, gas bottle, flow generator, and/or wall source. The gases flow to a patient interface (not shown in FIG. 3) via a wye connector 316. Expired gases then flow into the expiratory breathing tube 306B via the wye connector 316 and back to the gas source 312.

The inspiratory breathing tube 306A is pneumatically coupled to the chamber 304 via the intermediate tube section 314, which in this example is an elbow connector. An elbow connector may be particularly convenient to plug the connector 308 into, but the intermediate connector 314 could be of various other forms, for example straight or with a bend more or less than 90°. A control circuit of the heater base 302 is electrically connected to the inspiratory breathing tube 306A by connector 308. In this example, the control circuit is coupled to the inspiratory breathing tube 306A via the intermediate component 314. In particular, the connector 308 is mechanically connected to an electrical interface 318 of the intermediate component. The intermediate component includes electric lines providing electrical connection between the connector and the breathing tube.

As with the previous example, the breathing tube can include a heating element. Heating elements can be provided in one or both of the inspiratory breathing tube 306A and expiratory breathing tube 306B. A power supply can power the heating element(s) over power lines in the connector 308. The control circuit can control the power provided from the power supply to the heating element(s). The control circuit can also be in communication with one or more digital devices over a data line provided in the connector 308. For example, the electrical connector 308 includes a housing 310 intermediate its ends. The housing 310 can house a digital device. In one example, the digital device is a digital temperature sensor. This can be used to sense ambient temperature. Digital devices can be provided elsewhere in the humidifier system such as in the inspiratory breathing tube 306A, the expiratory breathing tube 306B, the chamber 304, the intermediate component 314 and/or the wye connector 316. The digital devices can include one or more flow sensor(s), pressure sensor(s), humidity sensor(s) and/or thermal imaging device(s) in addition to, or instead of, a temperature sensor.

FIG. 4 shows a portion of an electrical connector 400, which could serve as the connector 128 of FIGS. 1A and 1C, the connector 248 of FIG. 2 or the connector 308 of FIG. 3. This portion of the connector 400 includes the electrical interface 410. In this example, the interface 410 is in the form of a plug. The interface 410 could take other forms such as a socket (for receiving a plug), a jack, clips etc. In FIG. 4 the terminals are shown located in holes that are formed in projections at the distal end of the interface, however this is not limiting and the terminals could be located in other places on the interface such as at the side. The interface 410 has three terminals that correspond to the two power lines and the data line of the connector 400. These are the only terminals provided in this end of the connector. The terminals are a first power line terminal 402, a second power line terminal 404 and a data line terminal 406. The power line terminals 402 and 404 can provide power from a power supply to the breathing tube, for example to power a heating element. The data line terminal 406 can allow serial communications with one or more digital devices. As will be detailed further below, one of the power lines can also provide a reference potential for one or more digital devices.

The interface that the connector connects to (e.g. 144 of FIG. 1, 263 of FIG. 2 or 318 of FIG. 3) can be formed to correspond to this interface and be mechanically coupled to it. For example, if the interface of the connector 400 is in the form of a plug as shown in FIG. 4, the corresponding interface that it connects to would be a socket. The interface that the connector connects to also has only three terminals for connection to the three terminals of the connector 400.

An indicator in the form of a light-emitting diode (LED) can be provided under the sheath of the connector 400 at the interface 410 end as generally indicated by the dotted lines at 408. The indicator 408 can serve as a visual indicator of various operating, configurational or environmental conditions of the humidifier. It can also indicate other statuses of the humidifier such as whether it is turned on or off. The indicator 408 can also be located at other places on the connector 400. Instead of or in addition to the LED a different visual indicator such as a liquid crystal display or incandescent light element can be provided on the connector 400. The indicator 408 can be illuminated when a properly functioning tube heating element is connected to the heater base (alternatively, if inverse logic is employed then non-illumination could signal this). If a tube heating element is malfunctioning or not connected, the indicator 408 can indicate this. The indicator 408 can also indicate a fault or disconnection of the connector 400 (alternatively, if inverse logic is employed then non-illumination could signal this). A fault of the connector 400 can include a problem with the sensor(s) in the connector 400. The indicator can also indicate an environmental condition such as ambient temperature, for example it could indicate if the ambient temperature is above or below a specified range. The indicator 408 can indicate these conditions by, for example, being illuminated or unilluminated, by flashing, or by outputting a particular color of light. If the indicator 408 is configured to indicate more than one condition, it can have a number of different outputs, each corresponding to one of the conditions that it can indicate. For example, different colors, illumination patterns over time (i.e. flash sequences), or different combinations of lighting elements could indicate different conditions. The indicator 408 can act as a visual message or a visual warning.

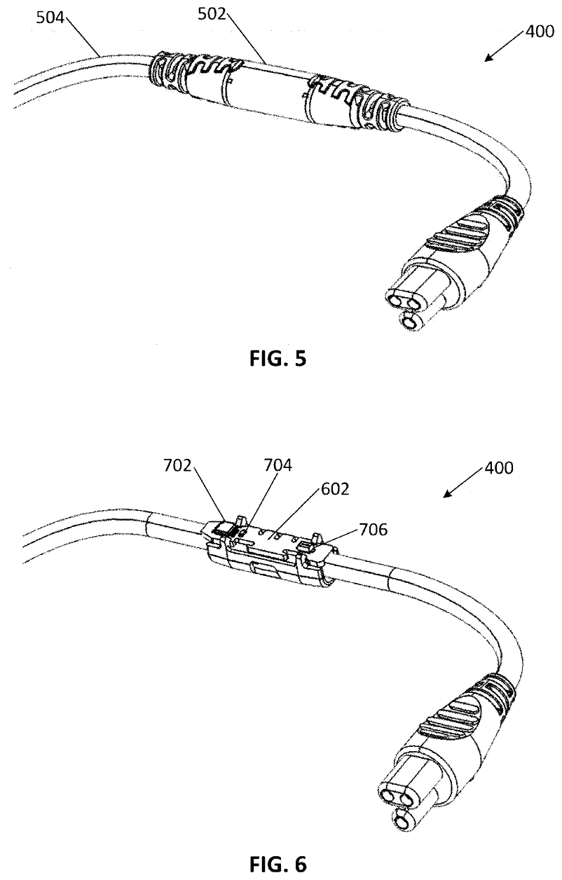

In the view of FIG. 5, the housing 502 and cable body 504 of the connector 400 can be seen. The housing 502 can house various components and electrical circuits such as one or more printed circuit boards (PCBs), sensors, digital devices, analog devices, LEDs, processing devices or data storage devices. The cable body 504 conducts electrical lines, for example in the form of wires, between the ends of the connector 400 and from one or both ends to components in the housing 502.

In FIG. 6, the outer layer of the housing has been removed from the connector 400, revealing the printed circuit board (PCB) 602. Also shown in FIG. 6 are the digital device (in the form of digital temperature sensor 702), analog temperature sensor 704 and a Zener diode 706. In this example, the digital temperature sensor 702, analog temperature sensor 704 and Zener diode are mounted on the same PCB 602. In other examples, these components may be mounted on two or more separate PCBs.

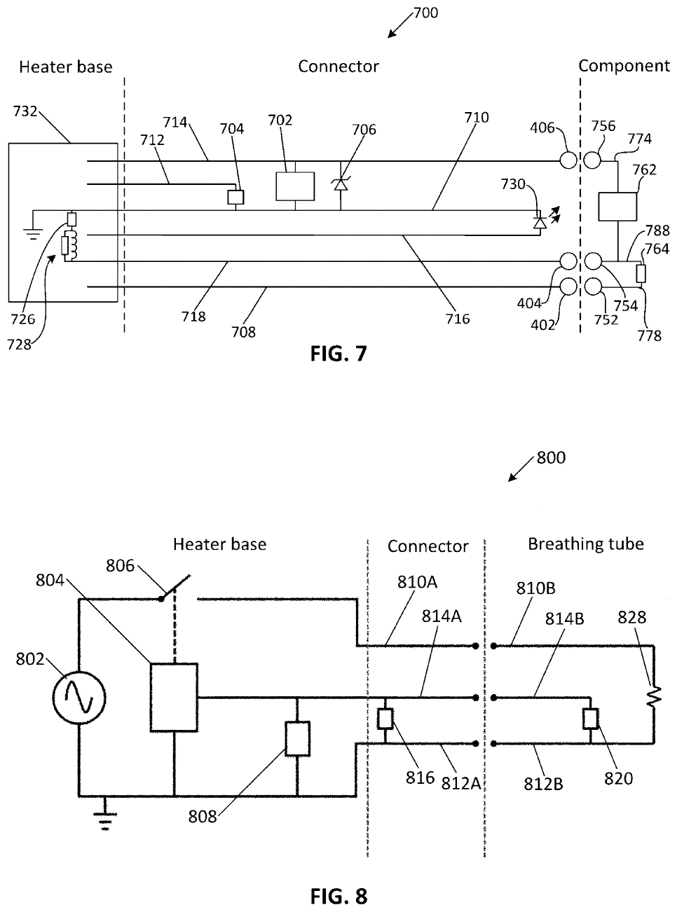

FIG. 7 shows an exemplary electrical circuit 700 of the connector, its connections to the heater base 732, and a component connected to the connector. The connector circuit 700 includes a digital temperature sensor 702, analog temperature sensor 704 and Zener diode 706. Extending from the heater base 732 are data line 714, an analog temperature sensor line 712, a ground line 710, an LED line 716 and two power lines 708 and 718. The data line 714, analog temperature sensor line 712 and ground line 710 can be soldered to respective pads of the PCB 602 of FIG. 6. The power lines 708 and 718 and the LED line 716 are not necessarily soldered to the PCB 602.

The analog temperature sensor line 712 runs from the heater base 732, through the cable body to the PCB and provides a voltage to the analog temperature sensor 704 which can include a thermistor in a voltage divider arrangement. The thermistor is located between the analog temperature sensor line 712 and the ground line 710, with the other resistive component(s) (not shown) of the voltage divider being located in the heater base 732 between a voltage source and the analog temperature sensor line 712. The analog temperature sensor line 712 can be connected to the control circuit in the heater base 732.

The ground line 710 runs from the heater base to the LED 730 at the end of the connector, with the LED 730 connected between the LED line 716 and the ground line 710.

The data line 714 runs from the heater base 732 to the interface at the breathing tube end of the connector and terminates in the data terminal 406 of the electrical interface provided on the connector. The digital temperature sensor 702 is connected between the data line 714 and the ground line 710. The data line 714 can be connected to the control circuit in the heater base 732. In the case that another digital device is used in place of, or in addition to, the digital temperature sensor 702, it could be provided with the same connections as digital temperature sensor 702 (i.e. between the data line 714 and the ground line 710).

The control circuit in the heater base can conduct serial communications with the digital device, in this case digital temperature sensor 702, over the data line 714. For example, the control circuit can interrogate the temperature sensor periodically to request a temperature reading. The digital temperature sensor 702 can report a sensed temperature over the data line 714. The control circuit can be arranged to receive an output indicative of temperature from the analog temperature sensor 704, in addition to or as an alternative to the digital temperature sensor 702. For example, a voltage across the analog temperature sensor 704 can be received at an analog pin of the control circuit. This can then be converted into a digital value by an analog-to-digital converter of the control circuit.

Ambient temperature may be sensed based on both temperature sensor readings. In one example, the readings from the digital and analog temperature sensors can be averaged to determine ambient temperature. The average may be weighted or unweighted. Alternatively, the reading from one sensor may be preferentially used, with the reading from the other sensor only used when the preferred sensor is not functioning correctly or provides a reading outside of an expected or predetermined range. The sensed ambient temperature may be used in a control algorithm to control the power supply to the heating element of the breathing tube. For example, the heater wire power could be set according to the following equation:

heater wire power=A*ambient temperature+B

where A and B are constants.

Similarly, temperature of the breathing gases to be supplied to the patient may be sensed based on readings from both an analog temperature sensor and a digital temperature sensor. In some examples, the gases temperature can be sensed using temperature sensors in a breathing tube, humidification chamber or patient interface. The sensed temperature can be used to control power to the heater wire of a breathing tube in one example.

Two sensors may provide redundancy in the case of failure of one. The use of two temperature sensors that use different fundamental principles of operation may provide improved reliability and redundancy. This is because the analog and digital temperature sensors are unlikely to fail in the same circumstances or in the same way because they are resistant to different adverse conditions and/or failure modes.

The use of two sensors allows faults in one or both of the sensors to be detected. For example, if the output of one sensor (or a value derived from it) differs from the output of the other sensor (or a value derived from it) by more than a difference threshold, this may indicate that one or both of the sensors is not working correctly. If the difference only exists briefly, this may just be a transitory anomaly and may not result in a fault detection. However, if this difference stays above the difference threshold for more than a time threshold, a fault condition can be determined. This fault determination can be performed by the control circuit in the heater base. Alternatively, a digital device such as a processing device in the housing could determine the fault condition and report it to the control circuit. The fault determination can employ more than one difference threshold and more than one time threshold. For example, a difference between the sensor outputs indicative of a low discrepancy in temperature readings (such as 2° C.) could be tolerated for a threshold period of time of several minutes, whereas a difference between the sensor outputs indicative of a higher discrepancy in temperature readings (such as 4° C.) could be tolerated for a threshold period of time of only tens of seconds. Upon determination of a fault, power to the heating element of the breathing tube and/or to the heater plate can be reduced or disabled. A log of sensor readings or differences between sensor values could also be stored on a device memory (volatile or non-volatile) and may indicate longer term trends towards failure.

The data line 714 allows for further digital devices to be coupled to the control circuit. For example, these can include sensors, processors or data storage devices in the chamber, in the breathing tube, or in a component intermediate the breathing tube and the patient interface or chamber. These digital devices can be located at various locations in the humidifier system.

The power lines 708 and 718 run from the heater base to the interface 410 at the breathing tube end of the connector 400. The first power line 708 terminates in the first power terminal 402 in the interface 410 such that it can electrically connect to the breathing tube, for example to the heating element of the breathing tube. The second power line 718 runs from the heater base through the connector to the interface 410 at the breathing tube end of the connector 400. The second power line terminates in the second power terminal 404 such that it can electrically connect to the breathing tube, for example to the heating element of the breathing tube. Power is provided to the breathing tube through the power lines 708 and 718, which are connected to a power source in the heater base 732 and have a voltage between them in use. The power source in the heater base can be an AC power source and the power lines 708 and 718 can have an AC voltage between them in use. Alternatively, a DC power source could be used.

One of the power lines, in this example the second power line 718, can be directly or indirectly connected to the ground line 710. Ground line 710 can be a digital ground line. Sensing circuitry can be connected to the second power line 718. In this example the second power line 718 is connected to ground line 710 via a transient current detector 728 and sensing resistor 726. The transient current detector 728 can include a resistor and an inductor connected in parallel with each other. Although the second power line 718 may not be directly connected to the ground line 710, it can provide a low impedance path to ground and common reference potential for the first power line 708 and the data line 714. This can enable digital devices outside of the connector to communicate over the data line 714 using the second power line 718 as a reference. In an alternative example to the one shown in FIG. 7, one or more digital devices in the connector (such as the digital temperature sensor 702) or outside the connector can connect to the second power line 718 to use it as a reference, rather than the ground line 710. For example, the Zener diode 706 and LED 730 may also connect to the second power line 718 instead of ground.

The LED line 716 runs from the heater base to the LED 730 at the breathing tube end of the connector, with the LED 730 connected between the LED line 716 and the ground line 710. This allows the LED 730 to be controlled by the control circuit of the heater base.

The Zener diode 706 provides protection against over voltage conditions. The Zener diode is connected between the data line and the ground line. If the voltage between these two lines exceeds the breakdown voltage of the Zener diode 706 it will conduct and provide a low-resistance path for current to flow between the data line and ground. This may help to avoid electrostatic discharge.

The power terminals 402 and 404 and data terminal 406 can connect to corresponding power terminals 752 and 754 and data terminal 756 of a component electrically connected to the connector, which for example can be a breathing tube, chamber, an intermediate connector with two electrical interfaces, or intermediate component of the breathing circuit. This can connect the power lines 708 and 718 to power lines 778 and 788 in the breathing tube, chamber or intermediate component and the data line 714 to a data line 774 in the breathing tube, chamber or intermediate component. The data line 774 in the attached component acts as an extension to the data line 714 through one or more components electrically connected to the connector.

The component electrically connected to the connector can have an electrical load 764. For example, when the component is a breathing tube the electrical load 764 can be a heating element of the breathing tube. The electrical load 764 is connected between the first power terminal 752 of the component and the second power terminal 754 of the component to receive power from a power source over the connector.

The component can also include a digital device 762, such as a temperature sensor for example. The digital device 762 can be connected to the data line 774 (which is an extension of data line 714) to communicate with a control circuit in the heater base 732, connector, or another location. In the configuration shown in FIG. 7, the digital device 762 in the component can use one of the power lines, in this case the second power line 788, as a reference for the data line 774. A Zener diode (not shown) can be connected in parallel to the digital device 762 to protect it from overvoltage conditions when line 718 (and therefore also line 788) varies from ground potential, for example due to voltage transients.

A simplified circuit showing the electrical connections over data, power and ground lines is set out in FIG. 8. This is an alternative implementation of the humidifier circuit from that of FIG. 7. For example, the circuit 800 of FIG. 8 differs from the circuit 700 of FIG. 7 in that it does not include the sensing resistor 726 and transient current detector 728. Put another way, the circuit 800 of FIG. 8 does not include a power line that is indirectly connected to a ground line. Instead, power line 812A is, or is directly connected to, a ground line, which can be a digital ground line, and serves as the reference for the data line. This circuit 800 may be simpler to construct, for example requiring less wiring and fewer connections to be made. Optionally, sensing components such as a current sensing resistor and/or transient current detector can be connected between the power line 810A and the power supply 802, for example in an arrangement similar to that of the sensing resistor 726 and transient current detector 728 of FIG. 7.

In the circuit 800, the heater base includes a power supply, in this case an AC power supply 802. Alternatively, a DC power supply could be used. The power supply 802 is connected to the power line 810A in the connector via the switch 806. The power line 810A couples to power line 810B of the breathing tube via terminals, forming a power line 810 common to both. For example, the terminal of the power line 810A can be provided in a plug like the one shown in FIG. 4, along with data and ground terminals, and can plug into a socket with corresponding terminals. The power line 810 powers a load 828 in the breathing tube, for example a heating element. Power to the load 828 is controlled by the switch 806. The control circuit in this example is a microcontroller 804. The microcontroller 804 can control the duty cycle of the switch 806 to control the power supplied to the load 828. The data line 814 has a portion 814A extending from the heater base that is extended by the data line 814B in the breathing tube, providing a data connection between the microcontroller 804 and one or more device(s) on the data line 814. This allows the microcontroller 804 to conduct serial communications with digital devices connected to the data line 814. The power line 812 has a portion 812A extending from the heater base that is extended by a power line 812B in the breathing tube, forming a power line 812 common to both. In this simplified example the power line 812 is also the digital ground and the components 808, 816, 820 and 828 are in turn connected directly to the digital ground. In other examples, these components could be indirectly connected to ground via one or more other components.

Three digital devices 808, 816 and 820 are shown connected between the data lines and the ground lines. The digital devices may not necessarily be powered by a dedicated power source or line. In this example, the digital devices are parasitically powered. For example, the digital devices can be powered over the data line 814. For example, the digital devices can be provided with a capacitor that draws current from the data line while the data line is held high. Several single-wire protocols which allow devices to be powered and communicate over the same line are known. For example, the digital devices could communicate with the microcontroller using “1-Wire” protocol by Maxim Integrated, or a local interconnect network (LIN) protocol. References herein to a 1-wire communication protocol include any suitable similar protocol, which may for example be referred to as a 1-wire, one-wire or single-wire protocol.

Other possible ways of powering the digital devices could be used instead. For example, the digital devices could be parasitically powered using devices that convert heat to electrical energy using a thermocouple, convert magnetic to electrical energy using a coil, convert radiofrequency (RF) electromagnetic energy to electric energy using an RF antenna, or convert mechanical vibrations to electric energy using a generator or micro-electromechanical system (MEMS) device.

The data line can be connected any number of digital devices, with the data line acting as a bus. In this example, the microcontroller 804 acts as a master device and the digital devices 808, 816 and 820 are slave devices. Although three slave devices are shown, more or fewer slave devices could be provided on the bus network. The slave device 816 in the connector can be the digital temperature sensor 702 shown in FIG. 7. The slave devices 808 and 820 can be a digital temperature sensor or other digital devices such as the other sensor types discussed previously. Other slave devices could exist in the connector.

Alternatively, one or more of the digital devices on the bus can be a slave microcontroller. The slave devices can be interrogated by the microcontroller 804 acting as a master. The slave device(s) can be coupled to another component such as a data storage device or an analog device.

The master device can selectively communicate with any number of the digital devices over the data line. For example, it may selectively communicate with two or more of the devices at a time. Each digital device can have an identifier (ID) that is used to address communications to one or more devices. The microcontroller can then uniquely address communications to any one or combination of digital devices on the bus.

The use of digital devices also allows the microcontroller to verify the integrity of communications. For example, the microcontroller can implement a cyclic redundancy check (CRC) on the communications. The microcontroller can also interrogate the sensors to determine their status, rather than only receiving temperature readings.

While several digital devices and sensors have been shown in various locations, such as at chamber or patient ends of the breathing tube and in intermediate components between the breathing tube and the chamber or patient interface, these are not necessarily required. In some examples, there are no sensors provided in these locations.

While the control circuit has been discussed in terms of a microcontroller in certain examples, it could take other forms. For example, the control circuit could include one or more microprocessors, field-programmable gate arrays (FPGA), complex programmable logic devices (CPLD), programmable array logic devices (PAL) or combinations thereof. The control circuit could be implemented in a single device or a combination of devices.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C-HF. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in non-volatile memory, such as an erasable programmable read-only memory (EPROM). It will be further appreciated that hardware modules may comprise connected logic units, such as gates, flip-flops and/or application specific integrated circuits, and/or may comprise programmable units, such as programmable gate arrays and/or processors. The modules described herein can be implemented as software modules, but also may be represented in hardware and/or firmware. Moreover, although in some embodiments a module may be separately compiled, in other embodiments a module may represent a subset of instructions of a separately compiled program and may not have an interface available to other logical program units.

In certain embodiments, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with users, operators, other systems, components, programs, and so forth.

It should be emphasized that many variations and modifications may be made to the embodiments described herein, the elements of which are to be understood as being among other acceptable examples. All such modifications and variations are intended to be included herein within the scope of this disclosure and protected by the following claims. Further, nothing in the foregoing disclosure is intended to imply that any particular component, characteristic or process step is necessary or essential.

What is claimed is:

1. A respiratory or surgical humidifier comprising:
a. a heater base configured to receive a humidifier chamber and supply humidified gases to a breathing tube when coupled with the chamber, the heater base comprising a control circuit; and
b. an electrical connector for electrically coupling the control circuit to a breathing tube, the electrical connector comprising:
i. a first power line;
ii. a second power line; and
iii. a data line configured to allow serial communication between the control circuit and one or more digital devices.

2. The respiratory or surgical humidifier of claim 1 wherein the one or more digital devices comprise one or more integrated circuits.

3. The respiratory or surgical humidifier of claim 1 or claim 2 wherein the one or more digital devices comprise one or more sensors.

4. The respiratory or surgical humidifier of claim 3 wherein the one or more sensors comprises one or more digital temperature sensors.

5. The respiratory or surgical humidifier of claim 4 further comprising one or more analog temperature sensors.

6. The respiratory or surgical humidifier of claim 4 or claim 5 wherein one or more of the digital temperature sensors and/or one or more of the analog temperature sensors is/are configured to sense an ambient temperature.

7. The respiratory or surgical humidifier of any one of claims 1 to 6 wherein one or more of the digital devices is located in the connector.

8. The respiratory or surgical humidifier of claim 7 wherein the connector comprises a first end, a second end and a housing intermediate the first end and the second end wherein one or more of the digital devices is located in the housing.

9. The respiratory or surgical humidifier of claim 8 when dependent on claim 5 or claim 6 wherein one or more of the analog temperature sensors is located in the housing.

10. The respiratory or surgical humidifier of claim 9 wherein one or more of the digital temperature sensors and one or more of the analog temperature sensors are provided on a common printed circuit board (PCB) in the housing.

11. The respiratory or surgical humidifier of claim 6 or any one of claims 7 to 10 when dependent on claim 6 wherein the control circuit is configured to determine ambient temperature using an output from the digital temperature sensor and an output from the analog temperature sensor.

12. The respiratory or surgical humidifier of claim 6 or any one of claims 7 to 11 when dependent on claim 6 wherein the control circuit is configured to control an amount of power delivered to the breathing tube over the power lines based on the determined ambient temperature.

13. The respiratory or surgical humidifier of claim 11 or claim 12 wherein the control circuit is configured to detect a fault when an output of the digital temperature sensor or a value derived from the output of the temperature sensor differs from an output of the analog temperature sensor or a value derived from the output of the analog temperature sensor by an amount greater than a first difference threshold for a period greater than a first time threshold.

14. The respiratory of surgical humidifier of claim 13 wherein the control circuit is configured to detect a fault when an output of the digital temperature sensor or a value derived from the output of the temperature sensor differs from an output of the analog temperature sensor or a value derived from the output of the analog temperature sensor by an amount greater than a second difference threshold for a period greater than a second time threshold.

15. The respiratory or surgical humidifier of any one of claims 1 to 14 wherein the one or more digital devices comprise one or more sensors selected from the group consisting of: a humidity sensor; a pressure sensor; an airflow sensor; a voltage sensor; a current sensor; a battery monitor; and a chemical sensor.

16. The respiratory or surgical humidifier of any one of claims 1 to 15 wherein the one or more digital devices comprise one or more processors or data storage devices.

17. The respiratory or surgical humidifier of any one of claims 1 to 16 wherein one or more of the digital devices is configured to be parasitically powered.

18. The respiratory or surgical humidifier of claim 17 wherein one or more of the digital devices that are parasitically powered are configured to receive electrical energy over the data line.

19. The respiratory or surgical humidifier of any one of claims 1 to 18 wherein the control circuit and digital devices are configured to communicate on the data line wherein the data line acts a bus.

20. The respiratory or surgical humidifier of claim 19 wherein the control circuit and digital devices are configured to communicate using a 1-wire communication protocol.

21. The respiratory or surgical humidifier of any one of claims 1 to 20 wherein one or more of the digital devices are located in the chamber, the breathing tube, and/or in an intermediate tube section between the chamber and the tube or between the tube and a patient interface.

22. The respiratory or surgical humidifier of any one of claims 1 to 21 wherein the connector comprises a Zener diode connected between the data line and the second power line for overvoltage protection.

23. The respiratory or surgical humidifier of any one or claims 1 to 22 wherein the connector is coupled to the control circuit by a wired connection.

24. The respiratory or surgical humidifier of any one of claims 1 to 22 wherein the connector is configured to be coupled to the control circuit by a plug.

25. The respiratory or surgical humidifier of any one of claims 1 to 22 wherein the connector is configured to wirelessly couple to the control circuit.

26. The respiratory or surgical humidifier of any one of claims 1 to 25 wherein the connector further comprises a variable visual indicator for indicating status information to a user.

27. The respiratory or surgical humidifier of claim 26 wherein the variable visual indicator comprises a light-emitting diode (LED).

28. The respiratory or surgical humidifier of claim 27 wherein the output of the visual indicator is controlled based on an operating, configurational or environmental condition of the humidifier.

29. The respiratory or surgical humidifier of claim 28 wherein controlling the output of the visual indicator comprises controlling the output color or temporal illumination pattern of the LED.

30. The respiratory or surgical humidifier of any one of claims 26 to 29 wherein the output of the visual indicator is controlled based on a fault condition of the humidifier.

31. The respiratory or surgical humidifier of claim 30 wherein the visual indicator is configured to produce a plurality different outputs corresponding to a plurality of respective fault conditions.

32. The respiratory or surgical humidifier of any one of claims 1 to 31 further comprising a component configured to be electrically connected to the connector, the component comprising:
a. a first power line configured to be electrically connected to the first power line of the connector;
b. a second power line configured to be electrically connected to the second power line of the connector; and
c. a data line configured to be electrically connected to the data line of the connector;
wherein the second power line of the component is configured to provide a reference for communications on the data line of the component.

33. The respiratory or surgical humidifier of claim 32 wherein the component is a breathing tube, an intermediate component of the breathing circuit, a humidifier chamber, or an intermediate connector.

34. The respiratory or surgical humidifier of any one of claims 1 to 33 wherein the connector has only three electrical terminals at an end that couples to the breathing tube, each terminal provided on a respective one of the first power line, second power line, and data line.

35. A respiratory or surgical humidifier system comprising:
a. a heater base comprising a control circuit;
b. a chamber configured to be received by the heater base;
c. a breathing tube configured to pneumatically couple with the chamber; and
d. an electrical connector for electrically coupling the control circuit to the breathing tube, the electrical connector comprising:
i. a first power line;
ii. a second power line; and
iii. a data line configured to allow serial communication between the control circuit and one or more digital devices.

36. The respiratory or surgical humidifier system of claim 35 wherein the one or more digital devices comprise one or more integrated circuits.

37. The respiratory or surgical humidifier system of claim 35 or claim 36 wherein the one or more digital devices comprise one or more sensors.

38. The respiratory or surgical humidifier system of claim 37 wherein the one or more digital devices comprise one or more temperature sensors.

39. The respiratory or surgical humidifier system of claim 37 or 38 wherein the one or more digital devices comprise one or more sensors selected from the group consisting of: a humidity sensor; a pressure sensor; an airflow sensor; a voltage sensor; a current sensor; a battery monitor; and a chemical sensor.

40. The respiratory or surgical humidifier system of any one of claims 35 to 39 wherein the one or more digital devices comprise one or more processors or data storage devices.

41. The respiratory or surgical humidifier system of any one of claims 35 to 40 wherein one or more of the digital devices is configured to be parasitically powered over the data line.

42. The respiratory or surgical humidifier system of any one of claims 35 to 41 further comprising an intermediate tube section configured to be pneumatically coupled to the breathing tube between the breathing tube and the chamber.

43. The respiratory or surgical humidifier system of any one of claims 35 to 42 further comprising an intermediate tube section configured to be pneumatically coupled to the breathing tube between the breathing tube and a patient interface.

44. The respiratory or surgical humidifier system of any one of claims 35 to 43 wherein the connector is configured to be mechanically connected to an electrical interface of the breathing tube.

45. The respiratory or surgical humidifier system of any one of claims 35 to 43 wherein the connector is configured to be mechanically connected to an electrical interface of the chamber.

46. The respiratory or surgical humidifier system of claim 42 wherein the connector is configured to be mechanically connected to an electrical interface of the intermediate tube section that is configured to be pneumatically coupled to the breathing tube between the breathing tube and the chamber.

47. The respiratory or surgical humidifier system of claim 43 wherein the connector is configured to be mechanically connected to an electrical interface of the intermediate tube section that is configured to be pneumatically coupled to the breathing tube between the breathing tube and the patient interface.

48. The respiratory or surgical humidifier of claim 44 wherein the connector is releasably connectable to the electrical interface of the breathing tube independently of pneumatic connections of the breathing tube.

49. The respiratory or surgical humidifier system of any one of claims 35 to 48 wherein the breathing tube is configured to simultaneously pneumatically couple to the chamber and electrically couple to the chamber, a patient interface, or an intermediate component connected between the breathing tube and the chamber or between the breathing tube and a patient interface.

50. The respiratory or surgical humidifier system of any one of claims 35 to 49 wherein the one or more digital devices comprises one or more temperature sensors in the connector.

51. The respiratory or surgical humidifier system of any one of claims 35 to 50 wherein the one or more digital devices comprises one or more temperature sensors in the breathing tube.

52. The respiratory or surgical humidifier system of any one of claims 35 to 51 wherein the one or more digital devices comprises one or more temperature sensors in the chamber.

53. The respiratory or surgical humidifier system of any one of claims 35 to 52 wherein the one or more digital devices comprises one or more temperature sensors in an intermediate component connected between the breathing tube and the chamber or between the breathing tube and a patient interface.

54. The respiratory or surgical humidifier system of any one of claims 50 to 53 wherein the breathing tube comprises a heating element and the control circuit is configured to control an amount of power provided to the heating element based on the output(s) of one or more of the temperature sensors.

55. The respiratory or surgical humidifier system of any one of claims 35 to 54 wherein the one or more digital devices comprises three digital devices and the control circuit is configured to selectively communicate with two or more of the three digital devices.

56. The respiratory or surgical humidifier system of claim 37 wherein there is no sensor provided at the end of the breathing tube configured to connect to a patient interface or in an intermediate component configured to be connected between the breathing tube and a patient interface.

57. The respiratory or surgical humidifier system of claim 37 or claim 56 wherein there is no sensor provided at the end of the breathing tube configured to connect to the chamber or in an intermediate component configured to be connected between the breathing tube and the chamber.

58. The respiratory or surgical humidifier system of any one of claims 35 to 57 wherein the one or more digital devices comprise one or more digital devices configured to communicate wirelessly with the control circuit or with each other.

59. The respiratory or surgical humidifier system of any one of claims 35 to 58 comprising a component configured to be electrically connected to the connector, the component comprising:
a. a first power line configured to be electrically connected to the first power line of the connector;
b. a second power line configured to be electrically connected to the second power line of the connector; and
c. a data line configured to be electrically connected to the data line of the connector;
wherein the second power line of the component is configured to provide a reference for communications on the data line of the component.

60. The respiratory or surgical humidifier of claim 59 wherein the component is the breathing tube, an intermediate component of the breathing circuit, a humidifier chamber, or an intermediate connector.

61. The respiratory or surgical humidifier system of any one of claims 35 to 60 wherein the connector has only three terminals at an end that couples to the breathing tube, each terminal provided on a respective one of the first power line, second power line, and data line.

62. A breathing tube for a respiratory or surgical humidifier, the breathing tube comprising:
a. a breathing conduit including a pneumatic coupling at one end to couple the breathing conduit with a humidifier chamber of a respiratory or surgical humidifier;
b. a heater element configured to heat gases within the breathing conduit; and
c. an electrical interface for electrically coupling the breathing tube to a heater base of a respiratory or surgical humidifier, the electrical interface comprising:
i. a first power line electrically connected to the heater element;
ii. a second power line electrically connected to the heater element; and
iii. a data line configured to allow serial communication between a control circuit in the heater base and one or more digital devices.

63. The breathing tube of claim 62 wherein one or more of the digital devices is located in the breathing tube.

64. The breathing tube of claim 63 wherein the one or more of the devices located in the breathing tube comprise an integrated circuit.

65. The breathing tube of claim 63 or 64 wherein the one or more of the devices located in the breathing tube comprise a temperature sensor.

66. The breathing tube of any one of claims 62 to 65 wherein one or more of the digital devices is located in a component that the breathing tube is configured to be coupled to.

67. The breathing tube of any one of claims 62 to 66 wherein the second power line is configured to provide a reference for communications on the data line.

68. The breathing tube of any one of claims 62 to 67 wherein the electrical interface has only three terminals, each of the terminals provided on a respective one of the first power line, the second power line, and the data line.

69. A respiratory or surgical humidifier comprising:
a. a heater base configured to receive a humidifier chamber and supply humidified gases to a breathing tube when coupled with the chamber, the heater base comprising a control circuit; and
b. an electrical connector for electrically coupling the control circuit to a breathing tube, the electrical connector comprising a data line configured to allow serial communication between the control circuit and one or more processing or data storage devices.

70. The respiratory or surgical humidifier of claim 69 wherein one or more of the processing or data storage devices are configured to be parasitically powered over the data line.

71. The respiratory or surgical humidifier of claim 69 or claim 70 wherein the connector has only three terminals at an end that is configured to couple to the breathing tube, one of the terminals provided on the data line, another of the terminals provided on a first power line and the other of the terminals provided on a second power line.

72. A respiratory or surgical humidifier system comprising:
a. a heater base comprising a control circuit;
b. a chamber configured to be received by the heater base;
c. a breathing tube configured to pneumatically couple with the chamber; and
d. an electrical connector for electrically coupling the control circuit and the breathing tube, the electrical connector comprising a data line configured to allow serial communication between the heater base and one or more processing or data storage devices.

73. The respiratory or surgical humidifier system of claim 72 wherein one or more of the processing or data storage devices are configured to be parasitically powered over the data line.

74. The respiratory or surgical humidifier system of claim 72 or claim 73 wherein the connector has only three terminals at an end that couples to the breathing tube, one of the terminals connected to the data line, another of the terminals connected to a first power line and the other of the terminals connected to a second power line.

75. A breathing tube for a respiratory or surgical humidifier, the breathing tube comprising:
a. a breathing conduit including a pneumatic coupling at one end to couple the breathing conduit with a humidifier chamber of a respiratory or surgical humidifier;
b. a heater element configured to heat gases within the breathing conduit;
c. one or more processing or data storage devices; and
d. an electrical interface for electrically coupling the breathing tube to a heater base of the respiratory or surgical humidifier, the electrical interface comprising a data line configured to allow serial communication between the one or more processing or data storage devices and a control circuit in the heater base.

76. The breathing tube of claim 75 wherein one or more of the processing or data storage devices is configured to be parasitically powered over the data line.

77. The breathing tube of claim 75 or claim 76 wherein the electrical interface has only three terminals, one of the terminals provided on the data line, another of the terminals provided on a first power line and the other of the terminals provided on a second power line.

78. A humidifier chamber configured to be received by a heater base of a respiratory or surgical humidifier, the humidifier chamber comprising:
a. a gases inlet for receiving gases to be humidified;
b. a gases outlet for supplying humidified gases to a breathing tube of the humidifier; and
c. one or more electrical lines for electrically coupling the heater base to the breathing tube, each electrical line having a terminal proximate the gases outlet;
wherein the one or more electrical lines are integral with the chamber.

79. The humidifier chamber of claim 78 wherein the one or more electrical lines comprise a data line configured to allow serial communications between a control circuit of the heater base and one or more digital devices.

80. The humidifier of claim 79 wherein the one or more electrical lines further comprise a first power line and a second power line.

81. An electrical connector for a respiratory or surgical humidifier, the electrical connector comprising:
a. a first electrical interface comprising two power terminals and a data terminal;
b. a second electrical interface comprising two power terminals and a data terminal, the power and data terminals of the second electrical interface being electrically connected to respective power and data terminals of the first electrical interface via two power lines and a data line; and
c. a digital device electrically connected to the data line and one of the power lines, the digital device configured to perform serial communications over the data line.
